(19) United States
(12) Patent Application Publication
(10) Pub. No.: US 2006/0094610 A1
(43) Pub. Date: May 4, 2006

(54) Cleansing composition

(75) Inventors: Naoya Yamato, Kawasaki-shi (JP); Masatoshi Saito, Kawasaki-shi (JP); Kazuhiko Tobita, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/267,512

(22) Filed: Nov. 7, 2005

Related U.S. Application Data
(63) Continuation of application No. PCT/JP04/06129, filed on Apr. 28, 2004.

(30) Foreign Application Priority Data
May 7, 2003 (JP) 129552/2003

(51) Int. Cl. A61K 8/00

(52) U.S. Cl. 510/130

(57) ABSTRACT

The present invention provides a body cleansing composition that contains (A) a long chain fatty acid or a salt thereof, (B) N-acyl-α-alanine N or a salt thereof, (C) a polyhydric alcohol which is liquid at room temperature, (D) a polymer compound and (E) an inorganic salt as essential components. The present invention also provides body cleansing composition that contains (A) a long chain fatty acid or a salt thereof, (B) N-acyl-α-alanine or a salt thereof, (C) a polyhydric alcohol which is liquid at room temperature and (F) an amphoteric surfactant as essential components. The body cleansing composition of the present invention lathers quickly during cleansing, is superior in the basic performance of cleansing agents such as lather amount, lather spreading, and lather retention, is free of a residual feeling or a frictional feeling during rinsing, and can give an appropriate moist feeling and a clarifying feeling after drying.

### CROSS-REFERENCE TO RELATED APPLICATIONS

The present application is a continuation of PCT/JP2004/006129, filed on Apr. 28, 2004, which claims priority to Japanese Application No. JP 2003-129552, filed on May 7, 2003, which are hereby incorporated by reference in their entirety.

### BACKGROUND OF THE INVENTION

1. Field of the Invention

The present invention provides a cleansing composition for the body of a subject in need thereof or a surface thereof, which contains at least one or more types of long-chain fatty acids or a salt thereof, N-acyl-α-alanine or a salt thereof, a polyhydric alcohol that is liquid at room temperature, and a polymer compound. More particularly, the present invention relates to a body cleansing composition, which quickly lathers during cleansing, has a superior lather amount compared to the basic performance of cleansing agents, has a superior lather spreading, has a superior lather retention, is free of a residual feeling or a frictional feeling during rinsing. In addition, the body cleansing composition of the present invention provides, after drying, a moist feeling while retaining a clarifying feeling.

2. Discussion of the Background

Conventionally, body cleansing agents for the skin employ cleansing components such as anionic surfactants represented by long chain fatty acid or a salt thereof (i.e., higher fatty acid soaps) and betaine-type amphoteric surfactants due to their high latherability and high cleansing performance. However, such body cleansing agents are defective in that they exhibit inferior lather quality, water rinsing performance and, with respect to feeling after drying, these body cleansing agents leave a residual feeling and a frictional feeling upon the skin during rinsing, and a dried-out feeling to the skin after drying.

Cationic polymer conditioners such as cationic cellulose have been as a cleansing agent to reduce friction during rinsing, imparting a conditioning effect and the like. However, when contained in a sufficiently large amount so as to afford the aforementioned effect, cationic polymers problematically degrade lather quality and leave a residual feeling during rinsing and after drying.

While glycerol, propylene glycol, sodium lactate and the like are known as moisturizers, body cleansing agents that simply combine these moisturizers with cleansing agents do not sufficiently improve a moist feeling of the skin.

JP-A-2001-19632 discloses a cleansing agent comprising N-acylalanine sodium salt, polyhydric alcohol such as glycerol, propylene glycol and the like and cocamidopropyl betaine. According to the study of the present inventors, however, such a cleansing agent results in an inferior lather quality and water rinsing properties, as well as an unsatisfactory clarifying feeling after drying.

JP-A-01-282299 discloses a cleansing agent containing a soap base and lauroyl-L-glutamic acid triethanolamine salt, and a cleansing agent containing a soap base and lauroylsarcosine triethanolamine salt. According to the study of the present inventors, however, these cleansing agents are defective in that they are inferior in lather properties such as lathering performance, lather amount and lather retention. In addition, these cleansing agents are generally unsatisfactory and provide inferior water rinsing properties, as well as clarifying feeling after drying.

JP-A-10-147800 discloses a cleansing agent containing a soap base, sodium N-lauroyl-L-glutamate, and glycerol, and a cleansing agent containing a soap base and cocoyl-N-methyl-β-alanine sodium salt. According to the study of the present inventors, however, this cleansing agent is defective in that it provide inferior lather properties such as lathering performance, lather amount and lather retention. In addition, these cleansing agents are generally unsatisfactory and provide inferior water rinsing properties, as well as a moist feeling or a clarifying feeling after drying.

JP-A-2002-338994 discloses a powder cleansing agent containing cocoyl-DL-alanine sodium salt, potassium myristate and O-[2-hydroxy-3-(trimethylammonio)propyl]dextran chloride. According to the study of the present inventors, however, this powder cleansing agent is defective in that it provides inferior water rinsing properties and clarifying feeling after drying, and is generally unsatisfactory.

JP-A-2002-293722 discloses a face wash containing lauroyl-DL-alanine triethanolamine salt, stearic acid, sodium carboxymethylcellulose, and 1,3-butylene glycol. According to the study of the present inventors, however, this face wash is defective in that it provides inferior lather retention and water rinsing properties. In addition, this face wash provides an inferior clarifying feeling after drying, and is generally unsatisfactory.

In view of the foregoing, there is a critical need for the development of a body cleansing composition, which lathers quickly during cleansing, is superior in the basic performance of cleansing agents of lather amount, lather spreading, and lather retention, is free of a residual feeling or a frictional feeling during rinsing, and gives, a moist feeling after drying while retaining a clarifying feeling.

### SUMMARY OF THE INVENTION

The present inventors have conducted intensive studies in an attempt to achieve the above-mentioned objects and found that by adding at least (D) a polymer compound and (E) an inorganic salt or at least (F) an amphoteric surfactant or (G) an alkali metal salt of acylamino acid (excluding the alkali metal salt for (B)) to (A) a long chain fatty acid or a salt thereof, (B) N-acyl-α-alanine or a salt thereof and (C) a polyhydric alcohol which is liquid at room temperature as basic ingredients, a cleansing agent capable of simultaneously achieving the basic performance of cleansing agents such as lathering, lather amount, lather spreading, and lather retention, which also exhibits superior feeling during rinsing and after drying can be realized.

Accordingly, it is an object of the present invention to provide

(1) A body cleansing composition, which contains the following (A), (B), (C), (D) and (E):
(A) a long chain fatty acid or a salt thereof;
(B) an N-acyl-α-alanine represented by the formula (I): 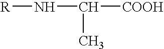
wherein R is a saturated or unsaturated acyl group having 8 to 26 carbon atoms, or a salt thereof;
(C) a polyhydric alcohol which is liquid at room temperature;
(D) a polymer compound; and
(E) an inorganic salt.

(2) A body cleansing composition, which contains the following (A), (B), (C) and (F):
(A) a long chain fatty acid or a salt thereof;
(B) an N-acyl-α-alanine represented by the formula (I): 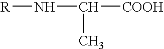
wherein R is a saturated or unsaturated acyl group having 8 to 26 carbon atoms, or a salt thereof;
(C) a polyhydric alcohol which is liquid at room temperature; and
(F) an amphoteric surfactant.

(3) A body cleansing composition, which contains the following (A), (B), (C) and (G):
(A) a long chain fatty acid or a salt thereof;
(B) an N-acyl-α-alanine represented by the formula (I): 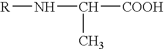
wherein R is a saturated or unsaturated acyl group having 8 to 26 carbon atoms, or a salt thereof;
(C) a polyhydric alcohol which is liquid at room temperature; and
(G) an alkali metal salt of acylamino acid (excluding the aforementioned alkali metal salt for (B)).

(4) The body cleansing composition of (3), further containing (F) an amphoteric surfactant.

(5) The body cleansing composition of any of (2)-(4), further containing (E) an inorganic salt.

(6) The body cleansing composition of any of (2)-(5), further containing (D) a polymer compound.

(7) The body cleansing composition of (1) or (6), wherein (D) a polymer compound is a cationic polymer.

(8) The body cleansing composition of any of (1)-(7), wherein (B) N-acyl-α-alanine or a salt thereof is N-acyl-L-alanine or a salt thereof.

(9) The body cleansing composition of any of (1)-(8), wherein (B) N-acyl-α-alanine or a salt thereof is an alkali metal salt of N-acyl-α-alanine.

(10) A body cleansing agent containing a cleansing composition of any of (1)-(9).

(11) A method of cleaning a body surface in a subject in need thereof comprising contacting the surface in need of such treatment with a cleansing composition of any of (1)-(10), lathering said cleansing composition, rinsing said composition from said surface, and drying said surface. In the method of (11), the surface in need of such treatment is skin or hair.

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, cosmetics, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

In the present invention, the phrase “body cleansing composition” means a cleansing composition to be used for the face and human body epidermis (skin) other than face, and the dosage form thereof includes various forms of liquid, lather, gel, solid, semi-solid, powder and the like.

The long chain fatty acid or a salt thereof for ingredient (A) to be used in the present invention is a straight chain or branched chain, saturated or unsaturated fatty acid or a salt thereof, and comprises single compounds having the same carbon number or a mixture of compounds having different carbon numbers.

As the basic component of the salt, the following may be used: alkali metals such as sodium, potassium and the like, alkaline earth metals such as magnesium, calcium and the like, organic amines such as monoethanolamine, diethanolamine, triethanolamine, 2-amino-2-methyl-1-propanol, 2-amino-2-methyl-1,3-propanediol and the like, inorganic amines such as ammonia and the like, basic amino acids such as lysine, ornithine, arginine and the like. Specific preference is given to alkali metals.

The long chain fatty acid or a salt thereof preferably has 8-26, particularly preferably 8-18, carbon atoms in the case of single compounds having the same carbon is number, and the average carbon atom number in the case of a mixture of compounds having different carbon atom numbers is preferably 8-26, particularly preferably 8-18. Specific preferable examples of the long chain fatty acid or a salt thereof include lauric acid, myristic acid, palmitic acid, stearic acid, coconut oil fatty acid, hydrogenated tallow fatty acid, palm oil fatty acid, hydrogenated palm oil fatty acid, safflower oil fatty acid, tallow fatty acid, behenic acid, oleic acid, isostearic acid and the like, and salts thereof can be mentioned. Of these, lauric acid (salt), myristic acid (salt), palmitic acid (salt), stearic acid (salt) and coconut oil fatty acid (salt) are particularly preferable.

The long chain fatty acid or a salt thereof for component (A) may be used alone or two or more kinds thereof may be used concurrently. While the amount thereof to be added is appropriately determined according to the object product (i.e. specific use of cleansing composition) and is not particularly limited, it is generally 0.1-80 wt %, preferably 0.5-30 wt %, more preferably 1-30 wt %, of the whole composition, from the viewpoints of lathering performance and lather amount.

N-acyl-α-alanine or a salt thereof to be used in the present invention, as component (B), comprises the aforementioned compound represented by the formula (I) or a salt, and may be N-acyl-D-alanine (salt), N-acyl-L-alanine (salt), N-acyl-DL-alanine (salt) or a mixture of two or more kinds selected from these. In the present invention, the compound of formula (I) is as follows:

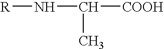

The saturated or unsaturated acyl group, which may be found in formula (I), has 8 to 26 carbon atoms for R in the formula (I) may be branched or straight chain and, for example, capryloyl group, caprinoyl group, lauroyl group, myristoyl group, palmitoyl group, stearoyl group, oleoyl group and the like can be mentioned.

In the present invention, the compound of the formula (I) may be a single compound wherein the acyl groups represented by R in the formula are the same, or a mixture of compounds wherein the acyl groups are different. As such mixture, compounds having an acyl group derived from naturally obtained mixed fatty, acids such as coconut oil fatty acid, tallow fatty acid, hydrogenated tallow fatty acid, castor oil fatty acid, olive oil fatty acid, palm oil fatty acid and the like or synthetically obtained fatty acids (including branched chain fatty acid) and the like can be mentioned. In the compound of the formula (I), R in the formula is preferably an acyl group derived from a mixed fatty acid, wherein the acyl group (i.e., R) has 8-26, preferably 8-18 carbon atoms on average, and particularly preferable acyl group derived from a mixed fatty acid is an acyl group derived from coconut oil fatty acid.

The basic component of a salt of the compound of the formula (I) may be an alkali metal (e.g., sodium, potassium etc.), an alkaline earth metal (e.g., magnesium, calcium etc.), an organic amine (e.g., monoethanolamine, diethanolamine, triethanolamine, 2-amino-2-methyl-1-propanol, 2-amino-2-methyl-1,3-propanediol etc.), an inorganic amine (e.g., ammonia etc.), or a salt comprising a basic amino acid (lysine, ornithine, arginine etc.) and the like. The salt is preferably an alkali metal salt from stability of the product form and the like, and a sodium salt is most preferable from the viewpoints of spreading of lather and lather retention.

In the present invention, N-acyl-α-alanine or a salt thereof for ingredient (B) is preferably N-acyl-DL-alanine or a salt thereof, and N-acyl-L-alanine or a salt thereof and N-acyl-L-alanine or a salt thereof are particularly preferable, in view of easy availability.

In addition, from the viewpoints of less residual feeling of the cleansing composition during rinsing and the like, use of N-acyl-L-alanine or a salt thereof is preferable.

Specific examples of N-acyl-α-alanine or a salt thereof that may be employed in the present invention as component (B) include N-cocoyl-DL-alanine sodium salt, N-cocoyl-L-alanine sodium salt, N-cocoyl-DL-alanine potassium salt, N-cocoyl-L-alanine potassium salt, N-cocoyl-DL-alanine triethanolamine salt, N-cocoyl-L-alanine triethanolamine salt, N-cocoyl-DL-alanine lysine salt, N-cocoyl-L-alanine lysine salt, N-cocoyl-DL-alanine arginine salt, N-cocoyl-L-alanine arginine salt, N-lauroyl-DL-alanine sodium salt, N-lauroyl-L-alanine sodium salt, N-lauroyl-DL-alanine potassium salt, N-lauroyl-L-alanine potassium salt, N-lauroyl-DL-alanine triethanolamine salt, N-lauroyl-L-alanine triethanolamine salt, N-lauroyl-DL-alanine lysine salt, N-lauroyl-L-alanine lysine salt, N-lauroyl-DL-alanine arginine salt, N-lauroyl-L-alanine arginine salt, N-myristoyl-DL-alanine sodium salt, N-myristoyl-L-alanine sodium salt, N-myristoyl-DL-alanine potassium salt, N-myristoyl-L-alanine potassium salt, N-myristoyl-DL-alanine triethanolamine salt, N-myristoyl-L-alanine triethanolamine salt, N-myristoyl-DL-alanine lysine salt, N-myristoyl-L-alanine lysine salt, N-myristoyl-DL-alanine arginine salt, N-myristoyl-L-alanine arginine salt, N-palmitoyl-DL-alanine sodium salt, N-palmitoyl-L-alanine sodium salt, N-palmitoyl-DL-alanine potassium salt, N-palmitoyl-L-alanine potassium salt, N-palmitoyl-DL-alanine triethanolamine salt, N-palmitoyl-L-alanine triethanolamine salt, N-palmitoyl-DL-alanine lysine salt, N-palmitoyl-L-alanine lysine salt, N-palmitoyl-DL-alanine arginine salt, N-palmitoyl-L-alanine arginine salt, N-stearoyl-DL-alanine sodium salt, N-stearoyl-L-alanine sodium salt, N-stearoyl-DL-alanine potassium salt, N-stearoyl-L-alanine potassium salt, N-stearoyl-DL-alanine triethanolamine salt, N-stearoyl-L-alanine triethanolamine salt, N-stearoyl-DL-alanine lysine salt, N-stearoyl-L-alanine lysine salt, N-stearoyl-DL-alanine arginine salt, N-stearoyl-L-alanine arginine salt, N-stearoyl-DL-alanine sodium salt, N-stearoyl-L-alanine sodium salt, N-stearoyl-DL-alanine potassium salt, N-stearoyl-L-alanine potassium salt, N-stearoyl-DL-alanine triethanolamine salt, N-stearoyl-L-alanine triethanolamine salt, N-stearoyl-DL-alanine lysine salt, N-stearoyl-L-alanine lysine salt, N-stearoyl-DL-alanine arginine salt, N-stearoyl-L-alanine arginine salt and the like. Of these, one wherein the acyl group is of a cocoyl type is preferable, and N-cocoyl-L-alanine sodium salt is particularly preferable.

In the present invention, N-acyl-α-alanine or a salt thereof (ingredient (B)) may be used alone or two or more kinds thereof may be used concurrently. While the amount thereof to be added is determined appropriately depending on the object product (i.e. specific use of cleansing composition) and is not particularly limited, it is generally 0.03-50 wt %, preferably 0.1-30 wt %, more preferably 1.0-25 wt %, of the whole composition.

In the present invention, a mixing ratio of long chain fatty acid or a salt thereof (component (A)) and N-acyl-α-alanine or a salt thereof (component (B)) is not particularly limited, but it is preferably 99/1-1/99, more preferably 50/50-1/99, particularly preferably 30/70-5/95.

As the polyhydric alcohol to be used as ingredient (C) in the present invention, which is liquid at room temperature, the following may be mentioned: ethylene glycol, diethylene glycol, triethylene glycol, propylene glycol, dipropylene glycol, polypropylene glycol, 1,3-butylene glycol, isoprene glycol, hexylene glycol, ethylhexanediol, polyethylene glycol, glycerol, diglycerol, polyglycerol and the like can be mentioned. Saccharides are not included as possible ingredient (C). Of these, propylene glycol, dipropylene glycol, 1,3-butylene glycol, hexylene glycol and glycerol are preferable. As used herein, room temperature means 20° C.

The polyhydric alcohol for ingredient (C) may be used alone or two or more kinds thereof may be used concurrently. While the amount thereof to be added is determined appropriately depending on the object product (i.e. specific use of cleansing composition) and is not particularly limited, it is generally 0.1-50 wt %, preferably 1-45 wt %, particularly preferably 5-40 wt %. When the amount thereof to be added is below 0.1 wt %, an unpreferable residual feeling during rinsing may occur or a sufficient moist feeling after drying sometimes may not be afforded. When the amount thereof to be added exceeds 50 wt %, the basic performance of cleansing agents of lather amount, lather spreading, and lather retention may be degraded or a clarifying feeling after drying may not be obtained.

The cleansing composition of the present invention comprises the above-described (A)-(C) as basic ingredients (basic components), and includes a composition (first embodiment) wherein at least (D) a polymer compound and (E) an inorganic salt were further added to 3 components of (A)-(C).

The cleansing composition of the present invention also encompasses a composition (second embodiment) wherein at least (F) an amphoteric surfactant is further added to 3 components of (A)-(C).

In addition, the cleansing composition of the present invention may be a composition (third embodiment) wherein at least (G) an alkali metal salt of acylamino acid (excluding alkali metal salt for (B)) is further added to 3 components of (A)-(C).

As is evident from the Examples and Comparative Examples (vide infra), with only 3 components of (A)-(C) a composition capable of simultaneously achieving the basic performance of cleansing agents such as lathering, lather amount, lather spreading, and lather retention and the feeling during rinsing and after drying, cannot be realized. Moreover, if any one of 3 components comprising the basic ingredients (i.e., ingredients (A)-(C)) is missing, even a composition wherein (D) a polymer compound and (E) an inorganic salt are further added to the remaining two components, or a composition wherein (F) an amphoteric surfactant or (G) an alkali metal salt of acylamino acid is further added to the remaining two components cannot simultaneously achieve the basic performance of cleansing agents such as lathering, lather amount, lather spreading, and lather retention and the feeling during rinsing and after drying, which is the object of the present invention.

Ingredient (D) to be used in the present invention is a polymer compound, which is not particularly limited except that it does not include polyalkylene glycol included in ingredient (C). Therefore, various polymer compounds such as anionic polymer, cationic polymer, amphoteric polymer and nonionic polymer and the like can be used in the present invention.

As an anionic polymer, for example, acrylic acid derivatives such as polyacrylic acid and a salt thereof (sodium, potassium, ammonium, triethanolamine, arginine, lysine salt etc.), acrylic resin, acrylic acid.acrylamide.ethyl acrylate copolymer and a salt thereof and the like; polyglutamic acid and a salt thereof; polyaspartic acid and a salt thereof; hyaluronic acid and a salt thereof; alginic acid and a salt thereof; methacrylic acid derivatives such as acrylic acid alkylester.methacrylic acid alkylester.diacetone.acetoneacrylamide.methacrylic acid copolymer and a salt thereof, polymethacrylic acid and a salt thereof and the like; crotonic acid derivatives such as vinyl acetate.crotonic acid copolymer, crotonic acid.vinyl acetate.vinyl neodecanate copolymer and the like; maleic acid derivatives such as methoxyethylene maleic anhydride copolymer, isobutylene.maleic acid copolymer and the like; carboxymethylcellulose and the like can be employed. Of these, acrylic acid derivatives such as polyacrylic acid and a salt thereof and the like, and carboxymethylcellulose are preferable.

As a nonionic polymer, for example, cellulose derivatives such as methylcellulose, hydroxypropylmethylcellulose, ethylcellulose, hydroxyethylcellulose, hydroxypropylcellulose and the like; vinyl derivatives such as polyvinyl alcohol, polyvinylpyrrolidone, polyvinyl butyral, vinyl pyrrolidone.styrene copolymer, vinylpyrrolidone.hexadecene copolymer, vinyl acetate.vinylpyrrolidone copolymer, eicosene.vinylpyrrolidone copolymer and the like; acrylic acid derivatives such as hydroxyethyl acrylate.methoxyethyl acrylate copolymer, hydroxyethyl acrylate.butyl acrylate.methoxyethyl acrylate copolymer, acrylic acid amide.styrene copolymer, alkyl acrylate.styrene copolymer, acrylic acid octylamide.acrylic acid ester copolymer, alkyl acrylate copolymer, acrylic acid octylamide.hydroxypropyl acrylate.methacrylic acid butylaminoethyl copolymer and the like; maleic acid derivatives such as vinylmethylether.ethyl maleate copolymer and the like; alginic acid propylene glycol; polyamide epichlorohydrin resin; polysaccharides such as guargum, locust bean gum, quince seed, carageenan, galactan, gum arabic, gum tragacant, pectin, mannan, starch, xanthan gum, dextran, curdlan, succinoglucan and the like and derivatives thereof; tallow derivatives such as polyoxyethylene tallow alkylhydroxymyristyreneether and the like; sorbitan derivatives such as polyoxyethylene sorbitan triisostearate and the like; sugar derivatives such as polyoxypropylene methyl glucose, polyoxyethylene methyl glucose dioleate and the like; and the like can be employed. Of these, cellulose derivatives such as methylcellulose, hydroxypropylmethylcellulose, ethylcellulose, hydroxyethylcellulose, hydroxypropylcellulose and the like are preferable.

As an amphoteric polymer, for example, copolymer such as acrylamide.acrylic acid.dimethyl diallylammonium chloride copolymer, acrylic acid.dimethyl diallylammonium chloride copolymer and the like; protein or hydrolyzed protein and derivatives thereof such as water-soluble collagen, hydrolyzed keratin and derivatives thereof, hydrolyzed silk and derivatives thereof, hydrolyzed gelatin and derivatives thereof, casein and a salt thereof, hydrolyzed casein and derivatives thereof, hydrolyzed conchiolin and derivatives thereof, hydrolyzed albumen and derivatives thereof, soybean proteolysis product and derivatives thereof, hydrolyzed wheat and derivatives thereof, hydrolyzed elastin and derivatives thereof, albumin and the like; succinylated carboxymethylchitosan and the like can be employed.

As the cationic polymer, chitin derivatives such as chitosan partially hydrolyzed chitin, chitosan dl-pyrrolidonecarboxylic acid salt, succinyl chitosan, hydroxypropyl chitosan and the like; dimethyl diallylammonium chloride derivative such as dimethyl diallylammonium chloride acrylamide copolymer (polyoctanium-7), polydimethyl methylene piperidinium chloride and the like; cationic cellulose such as O-[2-hydroxy-3-(trimethylammonio)propyl]hydroxyethylcellulose chloride, O-[2-hydroxy-3-(lauryl dimethylammonio)propyl]hydroxyethylcellulose chloride and the like; cationic guargum such as O-[2-hydroxy-3-(trimethylammonio)propyl]guargum chloride and the like; methacrylic acid derivatives such as methacryloylethyldimethyl betaine methacryloylethyltrimethylammonium chloride methoxy polyethylene glycol copolymer, methacryloylethyldimethyl betaine methacryloylethyltrimethylammonium chloride 2-hydroxyethyl methacrylate copolymer, vinylpyrrolidone N,N-dimethylaminoethylmethacrylic acid copolymer diethyl sulfate and the like; vinylimidazolinium methachloride.vinylpyrrolidone copolymer and the like can be employed, with preference given to cationic cellulose and cationic guargum.

In the present invention, one or more kinds of the polymer compound for ingredient (D) can be used, and at least a cationic polymer is preferably used. By the use of at least a cationic polymer, the feeling during rinsing and after drying is significantly enhanced.

The polymer compound for ingredient (D) may be used alone or two or more kinds thereof may be used concurrently. While the amount thereof to be added is determined appropriately depending on the object product (i.e. specific use of cleansing composition) and is not particularly limited, it is generally 0.01-15 wt %, preferably 0.05-10 wt %. When the amount thereof to be added is below 0.01 wt %, a frictional feeling may occur during rinsing, a sufficient moist feeling after drying may not be afforded. When the amount thereof to be added exceeds 15 wt %, the basic performance of cleansing agents of lather amount, lather spreading, and lather retention may be degraded, an unpreferable residual feeling may be caused during rinsing, and a clarifying feeling after drying may not be obtained.

Ingredient (E) to be used in the present invention is an inorganic salt, which is not particularly limited. However, a salt with mineral acids (sulfuric acid, hydrochloric acid, phosphoric acid and the like) or carbonic acid and alkali metal or alkaline earth metal are preferable. Specifically, sodium chloride, potassium chloride, magnesium chloride, calcium chloride, sodium sulfate, potassium sulfate, magnesium sulfate, calcium sulfate, sodium hydrogen sulfate, potassium hydrogen sulfate, monosodium phosphate, disodium phosphate, sodium carbonate, sodium hydrogen carbonate, potassium carbonate, potassium hydrogen carbonate, magnesium carbonate, calcium carbonate and the like can be employed. Of these, sodium chloride, potassium chloride, sodium sulfate and potassium sulfate are preferable.

The inorganic salts for ingredient (E) may be used alone or two or more kinds thereof may be used concurrently. While the amount thereof to be added is determined appropriately depending on the object product (i.e. specific use of cleansing composition) and is not particularly limited, it is generally 0.01-10 wt %, preferably 0.1-5 wt %, particularly preferably 0.1-3 wt %. When the amount thereof to be added is below 0.01 wt %, the basic performance of cleansing agents of lather amount, lather spreading, and lather retention may be degraded. In addition, a clarifying feeling after drying may not be obtained. When the amount thereof to be added exceeds 10 wt %, an unpreferable residual feeling may occur during rinsing, or an appropriate moist feeling after drying may not be obtained.

The amphoteric surfactant for component (F) to be used in the present invention is not particularly limited and various amphoteric surfactants can be used. Of these, carbobetaine type amphoteric surfactants, amidobetaine type amphoteric surfactants, sulfobetaine type amphoteric surfactants, hydroxysulfobetaine type amphoteric surfactants, amidosulfobetaine type amphoteric surfactants, phosphobetaine type amphoteric surfactants, imidazoline type amphoteric surfactants and the like are preferable, and amidobetaine type is particularly preferable. Specific examples thereof include lauryldimethylaminoacetic acid betaine, coconut oil alkyl betaine, stearyldimethylaminoacetic acid betaine, stearyldihydroxyethyl betaine, lauroylamidopropyl betaine, cocamidopropyl betaine, palm kernaloylamidopropyl betaine, ricinoleoyl amidopropyl betaine, N-cocoyl-N-carboxyethyl-N-hydroxyethylethylenediamine salt, palm kernaloyl-N-carboxyethyl-N-hydroxyethylethylenediamine salt, 2-alkyl-N-carboxymethyl-N-hydroxyethylimidazolium betaine, undecyl-N-carboxymethylimidazolinium betaine, alkyldiaminoethylglycine hydrochlorate liquid, bis(stearyl-N-hydroxyethylimidazoline)chloroacetic acid complex and the like.

The amphoteric surfactant for ingredient (F) may be used alone or two or more kinds thereof may be used concurrently. While the amount thereof to be added is determined appropriately depending on the object product (i.e. specific use of cleansing composition) and is not particularly limited, it is generally 0.1-30 wt %, preferably 0.5-20 wt %, more preferably 1-10 wt %. When the amount thereof to be added is below 0.1 wt %, an effect of improving the basic performance of cleansing agents of lather amount, lather spreading, and lather retention may not be obtained, or an appropriate moist feeling after drying may not be obtained sometimes. When the amount thereof to be added exceeds 30 wt %, unpreferable residual feeling or frictional feeling during rinsing may not occur or a clarifying feeling after drying may not be obtained.

Ingredient (G) to be used in the present invention is an acylamino acid alkali metal salt, which can be used without any limitation as long as it is an alkali metal salt of acylamino acid other than the alkali metal salt of N-acyl-α-alanine included in ingredient (B), and may be a salt of acylamino acid of a single compound having the same acyl group, or a salt of acylamino acid of a mixture of compounds having different acyl groups. Specifically, sodium cocoyl-methyltaurate, sodium methyl lauroyl taurate, sodium methyl myristoyl taurate, potassium cocoyl-methyltaurate, potassium methyl lauroyl taurate, potassium methyl myristoyl taurate, sodium cocoyl-taurate, sodium lauroyl taurate, sodium myristoyl taurate, potassium cocoyl-taurate, potassium lauroyl taurate, potassium myristoyl taurate, sodium cocoyl-L-glutamate, sodium lauroyl-L-glutamate, sodium myristoyl-L-glutamate, potassium cocoyl-L-glutamate, potassium lauroyl-L-glutamate, potassium myristoyl-L-glutamate, sodium cocoyl-methyl-β-alanine, sodium lauroylmethyl-β-alanine, sodium myristoylmethyl-β-alanine, potassium cocoyl-methyl-β-alanine, potassium lauroylmethyl-β-alanine, potassium myristoylmethyl-β-alanine and the like are preferable. Of these, a salt of cocoyl-amino acid wherein the acyl group is an acyl group derived from coconut oil fatty acid is particularly preferable.

One or more kinds of the amphoteric surfactant for ingredient (G) may be used. While the amount thereof to be added is determined appropriately depending on the object product (i.e. specific use of cleansing composition) and is not particularly limited, it is generally 0.1-30 wt %, preferably 0.5-20 wt %, more preferably 1-10 wt %. When the amount thereof to be added is below 0.1 wt %, an effect of improving the basic performance of cleansing agents of lather amount, lather spreading, and lather retention may not be obtained or an appropriate moist feeling after drying may not be obtained sometimes. When the amount thereof to be added exceeds 30 wt %, an unpreferable residual feeling or frictional feeling during rinsing may not occur or, a clarifying feeling after drying may not be obtained.

In the present invention, by adding an amphoteric surfactant for ingredient (F) or an alkali metal salt of acylamino acid for ingredient (G) to 3 components of ingredients (A)-(C) (basic ingredients), a cleansing agent capable of simultaneously achieving the basic performance of cleansing agents such as lathering, lather amount, spreading of lather and lather retention and a superior feeling during rinsing and after drying can be realized. Addition of both an amphoteric surfactant for ingredient (F) and an alkali metal salt of acylamino acid for ingredient (G) to 3 components of ingredients (A)-(C) (basic ingredients), more preferable results can be obtained.

As mentioned above, the cleansing composition of the present invention comprises ingredients (A)-(C) as basic ingredients (basic components). Further, (D) a polymer compound and (E) an inorganic salt may be added to 3 components of (A)-(C) (first embodiment), (F) an amphoteric surfactant may be added to 3 components of (A)-(C) (second embodiment), or (G) an alkali metal salt of acylamino acid (excluding acylalaninealkali metal salt for (B)) may be added to 3 components of (A)-(C) (third embodiment), whereby a cleansing agent achieving the object basic performance of cleansing agents and superior feeling during rinsing and after drying. In the compositions of the second and third embodiments, addition of at least one of (D) a polymer compound and (E) an inorganic salt is expected to afford preferable results, and the improving effect by the addition of (E) an inorganic salt to the composition of the third embodiment is particularly remarkable.

The cleansing composition of the present invention can contain concurrently with the recited components (A)-(G) in various combinations thereof, components such as oil generally used for cosmetic agents, powder (pigment, dye, resin), fluorine compound, resin, surfactant, adhesive, preservative, flavor, UV absorber, moisturizer, physiologically active component, solvent, antioxidant, antibacterial agent, adiaphoretic, chelating agent, neutralization agent, pH adjusting agent and the like, so long as the inclusion of said components does not inhibit the object (effect) of the present invention.

While the cleansing composition of the present invention can be used over a wide range from weak acidic to weak basic, its pH is preferably weak acidic of 5.0-8.0, particularly preferably 5.0-6.8, due to low irritation to the skin.

The cleansing composition of the present invention can be used as various body cleansing agents (i.e., cleansing agent to be used for face and human body epidermis (skin) other than face), and the dosage form thereof includes various forms of liquid, lather, gel, solid, semi-solid, powder and the like. As the specific use, face cleansing agent, makeup removing agents, facial foam, cleansing cream, body shampoo, hand soap, solid soap, shaving foam, shaving cream and the like can be mentioned.

As is evident from the following Examples, the present invention provides a cleansing composition that lathers quickly during cleansing, is superior in the basic performance of cleansing agents such as lather amount, spreading of lather and lather retention, free of a residual feeling or a frictional feeling during rinsing, and gives a moist feeling and a clarifying feeling after drying.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Production Example 1

### Synthesis of N-cocoyl-L-alanine sodium Salt

Water (167 g) was added to L-alanine (30.0 g), and cocoyl chloride (73.68 g) and 27 wt % aqueous NaOH solution were simultaneously added dropwise over 1 hr with stirring while maintaining pH of 10.98-11.02 and reaction temperature of 33-37° C. After aging for 1 hr, the reaction mixture was heated and 75 wt % sulfuric acid was added to adjust the pH to 2.0. The mixture was further heated to 80° C., stirred for about 10 min and incubated without mixing for 10 min to allow partitioning into an organic layer and an aqueous layer. The aqueous layer (about 200 g) was removed from the bottom part of a container. Hot water was added to the remaining organic layer, and the mixture was again heated to 80° C., stirred for about 10 min and incubated without mixing for 10 min. The partitioned aqueous layer was removed to give an organic layer. A 27% aqueous sodium hydroxide solution and water were added to the organic layer to adjust the pH to 6.8, whereby an object product (N-cocoyl-L-alanine sodium salt) was obtained.

### Production Example 2

### Synthesis of N-cocoyl-DL-alanine sodium Salt

In the same manner as in roduction Example 1 except that DL-alanine was used instead of L-alanine and, as such, the object product (N-cocoyl-DL-alanine sodium salt) was obtained.

### Examples 1-12

### Comparative Examples 1-9

The cleansing compositions (Examples 1-12, Comparative Examples 1-9) having the compositions shown in the following Tables 1 and 2 were prepared, and subjected to the following evaluation tests. In addition to the components listed in the table for each composition, the balance of the composition was distilled water, and the amount (numerical value in Tables) of each added component described in the Tables is weight fraction (%) when the whole composition is 100.

Evaluation Tests—

(i) Evaluation of the Basic Performance of the Compositions of Examples 1-12, Comparative Examples 1-9 to Serve as Cleansing Agents, Including Lathering Performance, Lather Amount, Lather Spreading, Lather Retention and the Like

Five professional panelists performed hand-washing evaluation. For evaluation, the average point obtained by the following grade table of not less than 4.1 was marked with ⊙, 3.5-4.0 was marked with ◯, 3.0-3.4 was marked with Δ, and not more than 2.9 was marked with X.

Lathering Performance
5: quick
4: rather quick
3: average
2: rather late
1: late

Lather Amount
5: thick
4: rather thick
3: average
2: rather insufficient
1: insufficient

Spreading of Lather
5: fine
4: rather fine
3: average
2: rather unpreferable
1: unpreferable

Lather Retention
5: fine
4: rather fine
3: average
2: rather unpreferable
1: unpreferable
   (ii) Functional Evaluation by Hand Washing Test

In the hand washing test, a residual feeling and a frictional feeling during rinsing, and a moist feeling and a clarifying feeling after drying were also evaluated by five professional panelists.

The evaluation was based on 5 levels of 1 point (bad) to 5 points (fine) and the average point of five panellers was calculated, wherein the average of not less than 4.1 was marked with ⊙, 3.5-4.0 was marked with ◯, 3.0-3.4 was marked with ▴, and not more than 2.9 was marked with X.

TABLE 1

| | Examples | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| stearic acid | 1 | 1 | 1 | 1 | 1 | 1 | | 1 | 1 | 1 | | 1 |
| potassium laurate | | | | | | | 5 | | | | 5 | |
| soap base *1 | | | | | | | 12 | | | | 12 | |
| sodium palm kernelate | | | | | | 25 | 11 | | | | 11 | |
| N-cocoyl-DL-alanine sodium salt | | 20 | | | | | | | | | | |
| N-cocoyl-L-alanine sodium salt | 20 | | | 20 | 10 | 7 | 5 | 20 | 12 | 12 | 5 | 20 |
| N-lauroyl-DL-alanine potassium salt | | | 20 | | | | | | | | | |
| glycerin | 30 | 30 | 30 | 30 | 30 | 20 | 25 | 30 | 30 | 30 | 25 | 30 |
| propylene glycol | 5 | 5 | 5 | 5 | 5 | 15 | | 5 | 5 | 5 | | 5 |
| Polyquaternium-7 *5 | 1 | 1 | 1 | 1 | 1 | 1 | | | | | | |
| carbomer *6 | 3 | 3 | 3 | 3 | 3 | | | | | | | |
| NaCl | 0.1 | 0.1 | 0.1 | 0.1 | 0.1 | 1 | 0.1 | | | | | 0.1 |
| Na₂SO₄ | 0.15 | 0.15 | 0.15 | 0.15 | 0.15 | | | | | | | 0.15 |
| citric acid | 0.7 | 0.7 | 0.7 | 0.7 | 0.7 | 0.7 | 0.7 | | | | | |
| cocamidopropyl betaine *7 | | | 3 | 3 | 3 | | 2 | 3 | | | 3 | 3 |
| N-cocoyl glutamic acid sodium salt *2 | | | | | | | | | | | | |
| N-cocoyl sarcosine sodium salt *3 | | | | | | | | | | | | |
| N-cocoyl N-methyl-β-alanine sodium salt *4 | | | | | | | | | | 10 | | |
| N-cocoyl methyltaurine sodium salt *3 | | | | | 10 | | | | 10 | | | |
| water | rest | rest | rest | rest | rest | rest | rest | rest | rest | rest | rest | rest |
| (basic performance) | | | | | | | | | | | | |
| lathering | ⊙ | ⊙ | ◯ | ⊙ | ⊙ | ⊙ | ⊙ | ⊙ | ⊙ | ⊙ | ⊙ | ⊙ |
| lather amount | ◯ | ◯ | ◯ | ◯ | ⊙ | ⊙ | ⊙ | ◯ | ⊙ | ⊙ | ⊙ | ⊙ |
| spreading of lather | ⊙ | ⊙ | ⊙ | ⊙ | ⊙ | ⊙ | ⊙ | ⊙ | ⊙ | ⊙ | ⊙ | ⊙ |
| lather retention | ◯ | ◯ | ◯ | ⊙ | ⊙ | ⊙ | ⊙ | ⊙ | ⊙ | ⊙ | ◯ | ⊙ |
| (skin) | | | | | | | | | | | | |
| residual feeling during rinsing | ⊙ | ◯ | ◯ | ⊙ | ⊙ | ⊙ | ⊙ | ◯ | ◯ | ◯ | ◯ | ⊙ |
| frictional feeling during rinsing | ⊙ | ⊙ | ⊙ | ⊙ | ⊙ | ◯ | ◯ | ◯ | ⊙ | ⊙ | ◯ | ◯ |
| moist feeling after drying | ◯ | ◯ | ◯ | ⊙ | ⊙ | ◯ | ⊙ | ⊙ | ◯ | ◯ | ⊙ | ⊙ |
| clarifying feeling after drying | ⊙ | ⊙ | ⊙ | ⊙ | ⊙ | ⊙ | ⊙ | ◯ | ⊙ | ⊙ | ◯ | ⊙ |

TABLE 2

| | Comparative Example 1 | | | | | | | | |
|---|---|---|---|---|---|---|---|---|---|
| | 1 | 2 | 3 | 4 | 5 | 6 | 7 | 8 | 9 |
| stearic acid | 1 | 1 | 1 | | 1 | 1 | 1 | 1 | |
| potassium laurate | | | | | | | | | 5 |
| soap base *1 | | | | | | | | | 12 |
| sodium palm kernelate | | | | | | | | | 11 |
| N-cocoyl-DL-alanine sodium salt | | | | | | | | | |
| N-cocoyl-L-alanine sodium salt | 20 | 20 | 20 | 20 | | | | | |
| N-lauroyl-DL-alanine potassium salt | | | | | | | | | |
| glycerin | | 30 | 30 | 30 | 30 | 30 | 30 | 30 | 25 |
| propylene glycol | | 5 | 5 | 5 | 5 | 5 | 5 | 5 | |
| Polyquaternium-7 *5 | 1 | | 1 | 1 | 1 | 1 | 1 | 1 | |
| carbomer *6 | 3 | | 3 | 3 | 3 | 3 | 3 | 3 | |
| NaCl | 0.1 | 0.1 | | 0.1 | 0.1 | 0.1 | 0.1 | 0.1 | 0.1 |
| Na₂SO₄ | 0.15 | 0.15 | | 0.15 | 0.15 | 0.15 | 0.15 | 0.15 | |
| citric acid | 0.7 | 0.7 | 0.7 | 0.7 | 0.7 | 0.7 | 0.7 | 0.7 | 0.7 |
| cocamidopropyl betaine *7 | 3 | | | 3 | 3 | 3 | 3 | 3 | 2 |
| N-cocoyl glutamic acid sodium salt *2 | | | | | 20 | | | | |
| N-cocoyl sarcosine sodium salt *3 | | | | | | 20 | | | 5 |
| N-cocoyl N-methyl-β-alanine sodium salt *4 | | | | | | | 20 | | |
| N-cocoyl methyltaurine sodium salt *3 | | | | | | | | 20 | |
| water | rest | rest | rest | rest | rest | rest | rest | rest | rest |
| (basic performance) | | | | | | | | | |
| lathering | ⊙ | ⊙ | ⊙ | Δ | Δ | Δ | Δ | ◯ | Δ |
| lather amount | ◯ | ◯ | ◯ | ◯ | Δ | ⊙ | ⊙ | ⊙ | ⊙ |
| spreading of lather | ⊙ | ⊙ | Δ | Δ | Δ | ◯ | ◯ | ◯ | ⊙ |
| lather retention | ⊙ | ⊙ | Δ | Δ | Δ | ⊙ | ⊙ | ⊙ | ⊙ |
| (skin) | | | | | | | | | |
| residual feeling during rinsing | ◯ | ⊙ | ⊙ | Δ | Δ | ◯ | ◯ | Δ | ⊙ |
| frictional feeling during rinsing | Δ | Δ | ◯ | ⊙ | ◯ | ◯ | ◯ | ◯ | ◯ |
| moist feeling after drying | Δ | Δ | ⊙ | ⊙ | ⊙ | Δ | Δ | ◯ | Δ |
| clarifying feeling after drying | ⊙ | ⊙ | Δ | ◯ | Δ | ⊙ | ⊙ | Δ | ⊙ |

In Tables 1, 2,
*1: tallow:coconut oil fatty acid = 8:2
*2: “Amisoft” CS-22 manufactured by Ajinomoto Co., Inc.
*3: “Soypon” SC manufactured by Kawaken Fine Chemicals Co., Ltd.
*4: “Alanon” ACE manufactured by Kawaken Fine Chemicals Co., Ltd.
*5: Marcoat Plus 2200 manufactured by Ondeo Nalco
*6: Carbopol EDT-2020 manufactured by BF Goodrich
*7: “Softazoline” CPB manufactured by Kawaken Fine Chemicals Co., Ltd.

As is evident from Tables 1 and 2, the cleansing compositions of Examples 1-12 are superior in all items of basic performance (lathering performance, lather amount, lather spreading, and lather retention), do not give a residual feeling or a frictional feeling during rinsing of the skin, give a moist feeling and a clarifying feeling to the skin after drying, and give a moist feeling and a clarifying feeling after drying.

### Examples 13-17

A body shampoo (i) (Example 13), body shampoo (ii) (Example 14), liquid hand soap (Example 15), facial foam (i) (Example 16) and facial foam (ii) (Example 17) were prepared by conventional methods according to the formulations shown in the following Tables. Each of these formulation are fine in the basic evaluation of lathering performance, lather amount, spreading of lather and lather retention, and also superior in feeling of use. The amount (numerical value in Table) of each added component described in each Table is weight fraction (%) when the whole composition is 100.

TABLE 3
(body shampoo (i))

| substances added | amount added (wt %) |
|---|---|
| water | 62.1 |
| propylene glycol | 6 |
| lauric acid | 5 |
| myristic acid | 4 |
| palmitic acid | 4 |
| N-cocoyl-L-alanine sodium salt | 3 |
| polyoxyethylenelauryl ether sulfate | 1 |
| potassium hydroxide (85%) | 3.6 |
| glycol distearate | 3 |
| glycerol | 3 |
| cocamidopropyl betaine | 3 |
| polyoctanium-39 | 0.5 |
| polyoctanium-7 | 0.5 |
| sodium chloride | 0.3 |
| etidronic acid | 0.3 |
| guarhydroxypropyl trimonium chloride | 0.2 |
| hydroxypropylcellulose | 0.2 |
| dibutylhydroxytoluene | 0.1 |
| paraben | 0.1 |
| edetate | 0.1 |
| flavor | suitable amount |

TABLE 4
(body shampoo (ii))

| substances added | amount added (wt %) |
|---|---|
| lauric acid | 2.0 |
| myristic acid | 2.0 |
| palmitic acid | 2.0 |
| N-cocoyl-DL-alanine sodium salt | 8.0 |
| PG | 6.0 |
| potassium hydroxide (85%) | 3.3 |
| water | 54.5 |
| lauramide MEA | 3.0 |
| cocamidopropyl betaine (30%) | 16.7 |
| glycol distearate *6 | 2.0 |
| sodium chloride | 0.3 |
| hydroxypropylmethylcellulose | 0.15 |

TABLE 5
(liquid hand soap)

| substances added | amount added (wt %) |
|---|---|
| water | 69.5 |
| potassium laurate | 6.0 |
| potassium myristate | 4.0 |
| potassium palmitate | 2.0 |
| propylene glycol | 3 |
| glycerol | 3 |
| N-lauroyl-DL-alanine potassium salt | 2 |
| cocamide DEA | 2 |
| glycol distearate | 1.5 |
| lauramidopropyl betaine | 1.5 |
| PEG-6 | 1.5 |
| hydroxypropylmethylcellulose | 1.5 |
| polyoctanium-39 | 1 |
| sodium chloride | 0.7 |
| edetate | 0.5 |
| benzoate | 0.2 |
| paraben | 0.1 |
| flavor | suitable amount |

TABLE 6
(facial foam (i))

| substances added | amount added (wt %) |
|---|---|
| water | 27.9 |
| potassium myristate | 10 |
| glycerol | 15 |
| myristic acid | 10 |
| potassium stearate | 10 |
| N-cocoyl-L-alanine sodium salt | 2 |
| glyceryl stearate (SE) | 2 |
| PEG-32 | 10 |
| PEG-6 | 5 |
| stearic acid | 2 |
| sodium cocoyl methyltaurate | 2 |
| lauramide DEA | 1 |
| castor oil | 0.5 |
| sodium phosphate | 0.5 |
| polyglycerol-6 | 0.5 |
| sodium lauroylglutamate | 0.4 |
| 1,3-butylene glycol | 1 |
| edetate | 0.2 |

TABLE 7
(facial foam (ii))

| substances added | amount added (wt %) |
|---|---|
| water | 28.4 |
| glycerol | 30 |
| N-cocoyl-L-alanine sodium salt | 25 |
| stearic acid | 1 |
| DPG | 5 |
| lauric acid | 1 |
| ceteth-20 | 1 |
| multitol | 2 |
| Na cocoyl methyltaurate | 5 |
| dimethicone | 0.5 |
| coconut oil | 0.5 |
| sodium sulfate | 0.2 |
| edetate | 0.3 |
| dibutylhydroxytoluene | 0.1 |

Numerous modifications and variations on the present invention are possible in light of the above teachings. It is, therefore, to be understood that within the scope of the accompanying claims, the invention may be practiced otherwise than as specifically described herein.

1. A body cleansing composition, which comprises:
(A) a long chain fatty acid or a salt thereof;
(B) an N-acyl-α-alanine represented by the formula (I): 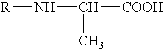
wherein R is a saturated or unsaturated acyl group having 8 to 26 carbon atoms, or a salt thereof;
(C) a polyhydric alcohol which is liquid at room temperature;
(D) a polymer compound; and
(E) an inorganic salt.

2. The body cleansing composition of claim 1, wherein (D) a polymer compound is a cationic polymer.

3. The body cleansing composition of claim 1, wherein (B) N-acyl-α-alanine or a salt thereof is N-acyl-L-alanine or a salt thereof.

4. The body cleansing composition of claim 1, wherein (B) N-acyl-α-alanine or a salt thereof is an alkali metal salt of N-acyl-α-alanine.

5. A body cleansing agent in a form selected from the group consisting of a liquid, a lather, a gel, a solid, a semi-solid, and a powder, wherein said body cleansing agent comprises a cleansing composition of claim 1.

6. A method of cleaning a body surface in a subject in need thereof comprising contacting the surface in need of such treatment with a cleansing composition of claim 1, lathering said cleansing composition, rinsing said composition from said surface, and drying said surface.

7. The method of claim 6, wherein said surface in need of such treatment is skin.

8. The method of claim 6, wherein said surface in need of such treatment is hair.

9. A body cleansing composition, which comprises:
(A) a long chain fatty acid or a salt thereof;
(B) an N-acyl-α-alanine represented by the formula (I): 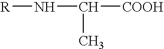
wherein R is a saturated or unsaturated acyl group having 8 to 26 carbon atoms, or a salt thereof;
(C) a polyhydric alcohol which is liquid at room temperature; and
(F) an amphoteric surfactant.

10. The body cleansing composition of claim 9, further comprising (E) an inorganic salt.

11. The body cleansing composition of claim 9, further comprising (D) a polymer compound.

12. The body cleansing composition of claim 11, wherein (D) a polymer compound is a cationic polymer.

13. The body cleansing composition of claim 9, wherein (B) N-acyl-α-alanine or a salt thereof is N-acyl-L-alanine or a salt thereof.

14. The body cleansing composition of claim 9, wherein (B) N-acyl-α-alanine or a salt thereof is an alkali metal salt of N-acyl-α-alanine.

15. A body cleansing agent in a form selected from the group consisting of a liquid, a lather, a gel, a solid, a semi-solid, and a powder, wherein said body cleansing agent comprises a cleansing composition of claim 9.

16. A method of cleaning a body surface in a subject in need thereof comprising contacting the surface in need of such treatment with a cleansing composition of claim 9, lathering said cleansing composition, rinsing said composition from said surface, and drying said surface.

17. The method of claim 16, wherein said surface in need of such treatment is skin.

18. The method of claim 16, wherein said surface in need of such treatment is hair.

19. A body cleansing composition, which comprises:
(A) a long chain fatty acid or a salt thereof;
(B) an N-acyl-α-alanine represented by the formula (I): 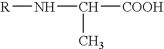
wherein R is a saturated or unsaturated acyl group having 8 to 26 carbon atoms, or a salt thereof;
(C) a polyhydric alcohol which is liquid at room temperature; and
(G) an alkali metal salt of acylamino acid, wherein said alkali metal salt of acylamino acid is not the alkali metal salt of (B).

20. The body cleansing composition of claim 19, further comprising (F) an amphoteric surfactant.

21. The body cleansing composition of claim 19, further comprising (E) an inorganic salt.

22. The body cleansing composition of claim 19, further comprising (D) a polymer compound.

23. The body cleansing composition of claim 22, wherein (D) a polymer compound is a cationic polymer.

24. The body cleansing composition of claim 19, wherein (B) N-acyl-α-alanine or a salt thereof is N-acyl-L-alanine or a salt thereof.

25. The body cleansing composition of claim 14, wherein (B) N-acyl-α-alanine or a salt thereof is an alkali metal salt of N-acyl-α-alanine.

26. A body cleansing agent in a form selected from the group consisting of a liquid, a lather, a gel, a solid, a semi-solid, and a powder, wherein said body cleansing agent comprises a cleansing composition of claim 19.

27. A method of cleaning a body surface in a subject in need thereof comprising contacting the surface in need of such treatment with a cleansing composition of claim 19, lathering said cleansing composition, rinsing said composition from said surface, and drying said surface.

28. The method of claim 27, wherein said surface in need of such treatment is skin.

29. The method of claim 27, wherein said surface in need of such treatment is hair.
